(19) United States
(12) Patent Application Publication
(10) Pub. No.: US 2016/0318972 A1
(43) Pub. Date: Nov. 3, 2016

(54) PREPARATION METHOD AND ANALYTIC METHOD FOR THE EXTRACT OF ANTRODIA CINNAMOMEA

(71) Applicant: YANG-CHANG WU, KAOHSIUNG CITY (TW)

(72) Inventors: YANG-CHANG WU, KAOHSIUNG CITY (TW); MEI-CHIN LU, PINGTUNG COUNTY (TW); TUNG-YING WU, KAOHSIUNG CITY (TW); YING-CHI DU, CHIAYI CITY (TW)

(73) Assignee: YANG-CHANG WU, KAOHSIUNG CITY (TW)

(21) Appl. No.: 15/141,916

(22) Filed: Apr. 29, 2016

(30) Foreign Application Priority Data
Apr. 30, 2015 (TW) 104113995

(51) Int. Cl. C07J 9/00

(52) U.S. Cl. CPC C07J 9/005

(57) ABSTRACT

An optimal preparation and analytic method for the extraction of Antrodia cinnamomea is disclosed. The important parameters for the extraction efficiency of the extract are analyzed using mathematic and statistical experimental designs, and the extract of A. cinnamomea and its particular triterpenoid compounds are quantified and identified using the quantitative NMR and HPLC-tandem MS. Whether the ergostane and lanostane triterpenoid compounds are contained in one drug, health food or other products and the total amounts and individual amounts therein can be analyzed, determined or quantified using the techniques disclosed herein.

### CROSS-REFERENCE TO RELATED APPLICATION AND CLAIM OF PRIORITY

This application claims the benefit of Taiwan Patent Application No. 104113995, filed on Apr. 30, 2015, at the Taiwan Intellectual Property Office, the disclosures of which are incorporated herein in their entirety by reference.

### FIELD OF THE INVENTION

The present invention is related to a preparation method and an analytic method for an extract of Antrodia cinnamomea. In particular, the present invention is related to an optimization of the preparation and analysis for the extract of A. cinnamomea, and an accurately-quantified analytic method for ergostane triterpenoid compounds and/or lanostane triterpenoid compounds in the extract of A. cinnamomea.

### BACKGROUND OF THE INVENTION

Antrodia cinnamemea (abbreviated hereinafter “AC”), by name niu-chang-chih or jang-jy, is an endemic fungus in Taiwan and grows on the inner wall of heartwood (or the dark/humid wood surface) of the particular Cinnamomum kanehirai at 400 to 2000 meters altitude. Therefore, it is very difficult to find the wide fruiting body of AC or identify the morphological appearance of this Aphyllophorales fungus. In addition, AC's price per unit weight is still high because its biologically active components have potential pharmaceutical values.

Because the fruiting body of AC is not easily found or artificially cultured, the mycelia products of AC are popular in the market and are marketed as having anticancer activity, and reduced treatment-related symptoms and other side effects. In addition, the mycelia products of AC have recently been reported to have anti-oxidant, antihypersensitive and immunostimulatory effects. It has been claimed that these mycelia products contain active components similar to wild fruiting bodies with cytotoxic triterpenoids, steroids, as well as immunostimulatory polysaccharides reported previously.

Traditionally AC has been used as a health food to prevent inflammation, hypertension, itchy skin and liver cancer. Therefore, the extracts of the mycelia and fruiting body of AC are deemed as a potential chemotherapeutic agent against hepatoma, prostate cancer, bladder cancer, lung cancer cells and so on. However, the activity mechanism and the anti-cancer ability of each active component have not been clarified and studied until now.

The triterpenoid components are AC's major secondary metabolites, which are also the most high-profile active components. At present, the quantitative criteria for evaluating the triterpenoid of AC are mostly divided into two methods. The first one is based on weight, i.e. the extractability, wherein ethanol (EtOH) is used as an extraction solvent, the various extraction time points are set as the control parameters, and the determined weight of the triterpenoid is evaluated as the extractability. However, the amount of total triterpenoids, i.e. ergostane triterpenoid and lanostane triterpenoid, and its individual amount in the obtained extract cannot be determined. The second method is spectrophotometry. The amount of the total triterpenoids of AC is determined using colormetry, which is based on a complex's color formed by reacting the specific functional group (i.e. carboxyl (—COOH)) of the triterpenoid of AC with a specific reagent under specific conditions.

In addition, there are double bonds in the molecular structure of most triterpenoids, and thus there are absorption peaks within the ultraviolet wavelength region. Therefore, the amount of total triterpenoids with similar absorption coefficient can be determined at the specific wavelength using the ultraviolet spectrometry. For instance, because oleanolic acid (a triterpenoid organic acid) and the ingredients of a sample have similar chemical structures, oleanolic acid at a variety of concentrations is reacted with a traditional developer (vanillin-glacial acetic acid-perchloric acid) to develop colors, and the sample also is reacted with the developer followed by determining the amount of the triterpenoid in the sample using colormetry. The detailed principle of color development is that the carboxyl group is dehydrated to create the structure of the double bond, and the double bond shift and bi-molecular condensation are carried out to generate a conjugated diene system, and then cations are formed under an acidic reaction to show the color. The method is an exclusive development method for the total triterpenoids. However, this method can easily cause false positives. If other ingredients in the sample are bound with a carboxyl group, the carboxyl group may react with a specific developing reagent and result in a misjudgement. The above two methods are the common methods for detecting the total triterpenoids in AC, but their accuracy is low. Both methods still have room for improvement if they are used as the standard of quantification. In addition, Taiwan Patent Publication No. TW 201416673 A discloses a quantitative method for the triterpenoids of Antrodia camphoata, wherein the fingerprint pattern of A. camphoata is obtained by chromatography, the integral area ratio of the absorption peak within a designated interval is compared to that of an external standard, followed by determining the amount of triterpenoids. However, Patent Publication No. TW 201416673 A does not disclose the extraction method of the sample, the total amounts of the regostane and lanostane triterpenoids in the sample, and the individual amounts. In addition, Taiwan Patent No. 1407100 discloses a method for analyzing the triterpenoid of A. camphoata using high performance liquid chromatography (HPLC). However, it does not disclose a quantitative method of the triterpenoid included in the fruiting body of A. camphoata.

It is therefore the Applicant's attempt to deal with the above limitations in the prior art.

### SUMMARY OF THE INVENTION

To overcome the problems that the extractability of an extract of AC and its specific triterpenoid compounds cannot be efficiently quantified in the prior art, and to establish an optimal procedure for extracting AC so as to certify and control the amount of total triterpenoids included, in the present invention, the beginning of the extraction procedure for AC is analyzed, the accurate quantitative standard for triterpenoids is developed to ensure and control the quality of the raw material “AC”. In the present invention, the important parameters that may influence the extractability of the extract are analyzed using experimental designs from mathematics and statistics, and the extract of AC and its specific triterpenoid compounds are quantified and identified using the quantitative method of the nuclear magnetic resonance (NMR) spectrum and HPLC-tandem mass spectrometry (HPLC-tandem MS). Based on the above techniques, whether there are ergostane and/or lanostane triterpenoid compounds included in a medicine, a health food or other products or their total amount and their individual amounts can be analyzed/detected/quantified.

The term “the extract of AC” herein refers to an extract extracted from the fruiting body of AC, the mycelia of AC or the combination thereof, i.e. the extract of the fruiting body of AC, the extract of the mycelia of AC or the extract of the fruiting body and mycelia of AC. Preferably, the extract herein refers to an extract obtained by extracting the fruiting body of AC, the mycelia of AC or the combination thereof with an alcohol. The alcohol herein preferably is methanol (MeOH), ethanol (EtOH) or the combination thereof. Preferably, the concentration of the alcohol, MeOH and/or EtOH herein is a concentration caused by formulating the alcohol, MeOH and/or EtOH with water. The terms “Antrodia camphorata”, “AC” and “the extract of AC” herein contain the ergostane triterpenoids and/or lanostane triterpenoids, which are collectively named triterpenoids. All kinds of the ergostane triterpenoids and the lanostane triterpenoids respectively are collectively named as the total ergostane triterpenoids and the total lanostane triterpenoids, which are further collectively named as the total triterpenoids. In addition, single-formula medicines, complex-formula medicines, prescription medicines, non-prescription medicines, health foods, drinks or the like which actually contain or are announced to contain the ergostane triterpenoids and the lanostane triterpenoids fall into the definition of the term “the extract of AC” in the present invention.

The present invention discloses a pharmaceutical composition, which includes an efficient dosage of at least one compound of the ergostane triterpenoids (formulas I to X) and the lanostane triterpenoids (formulas XI to XIV) described as follows.

The ergostane triterpenoid compounds may include antcin K (formulas I and II), antcin C (formulas III and IV), zhankuic acid C (formulas V and VI), zhankuic acid B (formula VII), zhankuic acid A (formulas VIII and IX) and/or antcin A (formula X) (which are referred to as stereoisomeric pure compounds), and the lanostane triterpenoid compounds may include dehydrosulphurenic acid (formula XI), sulphurenic acid (formula XII), dehydroeburicoic acid (formula XIII) and/or eburicoic acid (formula XIV).

The present invention discloses a method for detecting the amount of at least one ergostane triterpenoid compound in AC, including steps of: extracting the fruiting body and/or mycelia of AC with an alcohol to obtain an extract of AC; and detecting the extract of AC using ¹H NMR to identify whether at least one ergostane triterpenoid compound is present in the extract of AC.

Furthermore, the detection step further includes: detecting a C-28 methylene signal of the at least one ergostane triterpenoid compound using ¹H NMR.

Additionally, the detection method is further used to detect the amount of at least one lanostane triterpenoid compound at the same time, and the method includes steps of: detecting the extract of AC using ¹H NMR to determine whether at least one lanostane triterpenoid compound is present in the extract of AC; and detecting a C-28 methylene signal of the at least one lanostane triterpenoid compound using ¹H NMR.

The present invention further discloses a method for detecting a total amount of the ergostane triterpenoid compound in an extract, including steps of: preparing standards at a variety of concentrations using a specific ergostane triterpenoid compound, performing the ¹H NMR spectrum for the standards and calculating a calibration curve of the standards; analyzing a C-28 methylene signal of the ergostane triterpenoid compound in the extract using ¹H NMR; and determining an integral area ratio for the C-28 methylene signal from the calibration curve, and calculating the total amount of the ergostane triterpenoid compound in the extract.

In accordance with the detection method, the present invention further discloses a method for detecting a total amount of the lanostane triterpenoid compound in an extract, including steps of: preparing standards at a variety of concentrations using a specific lanostane triterpenoid compound, performing the ¹H NMR spectrum for the standards and calculating a calibration curve of the standards; analyzing a C-28 methylene signal of the lanostane triterpenoid compound in the extract using ¹H NMR; and determining an integral area ratio for the C-28 methylene signal from the calibration curve, and calculating the total amount of the lanostane triterpenoid compound in the extract.

Furthermore, the method for detecting the total amounts of the ergostane triterpenoid compound and the lanostane triterpenoid compound in the extract further includes steps of: using pyrazine as an internal control for the ¹H NMR spectrum; detecting whether there is a C-28 methylene signal in the extract at δ_{H} 4.82 (2H, br d); and detecting whether there is a C-28 methylene signal in the extract at δ_{H} 4.63 (1H, s) and δ_{H} 4.70 (1H, s). The presence of the C-28 methylene signal at δ_{H} 4.82 (2H, br d), δ_{H} 4.63 (1H, s) and δ_{H} 4.70 (1H, s) indicates that the extract contains at least one ergostane triterpenoid compound and at least one lanostane triterpenoid compound.

The present invention further discloses a method for extracting at least one ergostane triterpenoid compound and at least one lanostane triterpenoid compound in AC, including steps of: providing the fruiting body and/or mycelia of AC and grinding it into fine powder; performing the extraction procedure by designing the different extraction parameters and using an alcohol as the extraction solvent to obtain an alcohol extract of the fruiting body and/or mycelia of AC, wherein the extract includes at least one ergostane triterpenoid compound (stereoisomeric pure compound) and at least one lanostane triterpenoid compound.

Preferably, the extraction method further includes: setting up a first parameter (temperature), a second parameter (time) and a third parameter (such as the concentration of EtOH) as the individual independent extraction parameters; determining an integral area value (i.e. an extraction response value) of the C-28 methylene signal of the ergostane triterpenoid compound and the lanostane triterpenoid compound of the extract using NMR; performing the extraction by designing the various extraction parameters, and analyzing the individual independent parameters or the interrelationship among the parameters and their significance, to obtain an adequate set of extraction parameters for at least one ergostane triterpenoid compound and at least one lanostane triterpenoid compound; and calculating the variations between the independent extraction parameters and the extraction response values by the multiple regression analysis as in the following formula I:

y=A₀+A₁x₁+A₂x₂+A₃x₃+A₁₂x₁x₂+A₁₃x₁x₂+A₂₃x₂x₃+A₁₁x₁²+A₂₂x₂²+A₃₃x₃² (I),

where y represents the extraction response value, A(0, 1, 2, 3, 12, 13, 23, 11, 22, 23) represent respective constants, and x(1, 2, 3) represent the independent control parameters.

The present invention further discloses a method for detecting the individual amount of the at least one ergostane triterpenoid composition in an extract, including steps of: extracting the fruiting body, mycelia or a mixture of the fruiting body and mycelia of AC using the alcohol to obtain an alcohol extract; detecting the alcohol extract using ¹H NMR to identify whether there is at least one ergostane triterpenoid compound in the alcohol extract; and detecting the amount of the at least one ergostane triterpenoid compound (stereoisomeric pure compound) in the alcohol extract using HPLC when the at least one ergostane triterpenoid compound is present.

Furthermore, the detection method further is used to detect the individual amount of the at least one lanostane triterpenoid compound at the same time, including steps of: detecting the alcohol extract using ¹H NMR to identify whether there is at least one lanostane triterpenoid compound in the alcohol extract; and detecting the amount of the at least one lanostane triterpenoid compound in the alcohol extract using HPLC when the at least one lanostane triterpenoid compound is present. A full wavelength detector and/or a detector composed of the full wavelength detector and a tandem mass spectrometer are used in the HPLC technique.

Furthermore, the detection method further includes: calculating the pKa values of the at least one ergostane triterpenoid compound and the at least one lanostane triterpenoid compound; adjusting the pH value of mobile phase for isolation; analyzing the at least one ergostane triterpenoid compound and the at least one lanostane triterpenoid compound in the same chromatographic spectrum.

Furthermore, the detection method further includes: chromatographing the alcohol extract and the standards corresponding to the at least one ergostane triterpenoid compound and the at least one lanostane triterpenoid compound; comparing an HPLC spectrum of the alcohol extract with those of the standards; analyzing a parent ion (pseudo-ion peak), a daughter ion at the first strength and a daughter ion at the second strength of the at least one ergostane triterpenoid compound and the at least one lanostane triterpenoid compound using the tandem mass spectrometer so as to determine whether there is the at least one ergostane triterpenoid compound and the at least one lanostane triterpenoid compound in the alcohol extract when the at least one ergostane triterpenoid compound and the at least one lanostane triterpenoid compound appear on the HPLC spectrum of the alcohol extract; preparing the standards at a variety of concentrations and calculating a calibration curve for the standards; analyzing the signals of the standards for the at least one ergostane triterpenoid compound and the at least one lanostance triterpenoid compound in the alcohol extract using the tandem mass spectrometer; and comparing the calibration curves of the standards to obtain the integral area ratio, and calculating the individual amounts of the at least one ergostane triterpenoid compound and the at least one lanostane triterpenoid compound in the alcohol extract.

According to the concept of the present invention, the present invention can be used to detect the total amounts of the at least one ergostane triterpenoid compound and the at least one lanostane triterpenoid compound in a complex-formula medicine and their individual amounts, the total amount of the at least one ergostane triterpenoid compound and the at least one lanostane triterpenoid compound in a single-formula medicine and their individual amounts, or whether there is at least one of the above two types of compounds in another herbal medicine sample or the total amounts of the specific compounds in at least one of two types of compounds and their individual amounts. Furthermore, the ratio of these two types of compounds or the specific compounds in two types of compounds in the complex-formula medicine, the single-formula medicine or other herbal medicine are determined.

### BRIEF DESCRIPTION OF THE DRAWINGS

The objectives and advantages of the present invention will become more readily apparent to those ordinarily skilled in the art after reviewing the following detailed descriptions and accompanying drawings.

FIG. 1 is an ¹H NMR spectrum (DMSO-d6, 400 MHz) of the total triterpenoids in the EtOH extract of AC which is extracted via heat reflux.

FIG. 2 is an ¹H NMR spectrum (DMSO-d6, 400 MHz) of the total triterpenoids in the EtOH extract of AC which is extracted using ultrasonic oscillation.

FIG. 3 is a diagram showing that the expectation values for the total ergostane triterpenoid compounds and the total lanostane triterpenoid compounds under the optimal extraction conditions.

FIGS. 4(A) and 4(B) are diagrams showing the optimal HPLC spectra of the EtOH extract of the fruiting body of AC in the present invention.

FIG. 5 illustrates the structural formula of the internal standard, i.e. ganoderic acid A, for the HPLC-MS in the present invention.

### DETAILED DESCRIPTION OF THE PREFERRED EMBODIMENTS

The present invention will now be described more specifically with reference to the following embodiments. It is to be noted that the following descriptions of preferred embodiments of this invention are presented herein for purpose of illustration and description only; they are not intended to be exhaustive or to be limited to the precise form disclosed.

The TUPAC nominations and the structural formulas of the ergostane triterpenoid compounds E1 to E12 (formulas I to X) extracted in the present invention are listed below in detail.

| | | Struc- | |
|---|---|---|---|
| Ergostane | | tural | |
| triterpenoid | Source | formula | IUPAC nomination |
| E1 | antcin | I | 3α,4β,7β-trihydroxy-4α- |
| | K | | methylergosta-8,24(28)-dien-11- |
| | | | on-25S-26-oic acid |
| E2 | | II | 3α,4β,7β-trihydroxy-4α- |
| | | | methylergosta-8,24(28)-dien-11- |
| | | | on-25R-26-oic acid |
| E3 | antcin | III | 7β-hydroxy-4α-methylergosta- |
| | C | | 8,24(28)-dien-3,11-dion-25S-26- |
| | | | oic acid |
| E4 | | IV | 7β-hydroxy-4α-methylergosta- |
| | | | 8,24(28)-dien-3,11-dion-25R-26- |
| | | | oic acid |
| E5 | zhankuic | V | 3α,12α-dihydroxy-4α-methylergosta- |
| | acid C | | 8,24(28)-dien-7,11-dion-25R-26- |
| | | | oic acid |
| E6 | | VI | 3α,12α-dihydroxy-4α-methylergosta- |
| | | | 8,24(28)-dien-7,11-dion-25S-26- |
| | | | oic acid |
| E7 | zhankuic | VII | 3α-hydroxy-4α-methylergosta- |
| E8 | acid B | | 8,24(28)-dien-7,11-dion-26-oic acid |
| E9 | zhankuic | VIII | 4α-methylergosta-8,24(28)-dien- |
| | acid A | | 3,7,11-trion-25S-26-oic acid |
| E10 | | IX | 4α-methylergosta-8,24(28)-dien- |
| | | | 3,7,11-trion-25R-26-oic acid |
| E11 | antcin | X | 4α-methylergosta-8,24(28)-dien- |
| E12 | A | | 3,11-dion-26-oic acid |

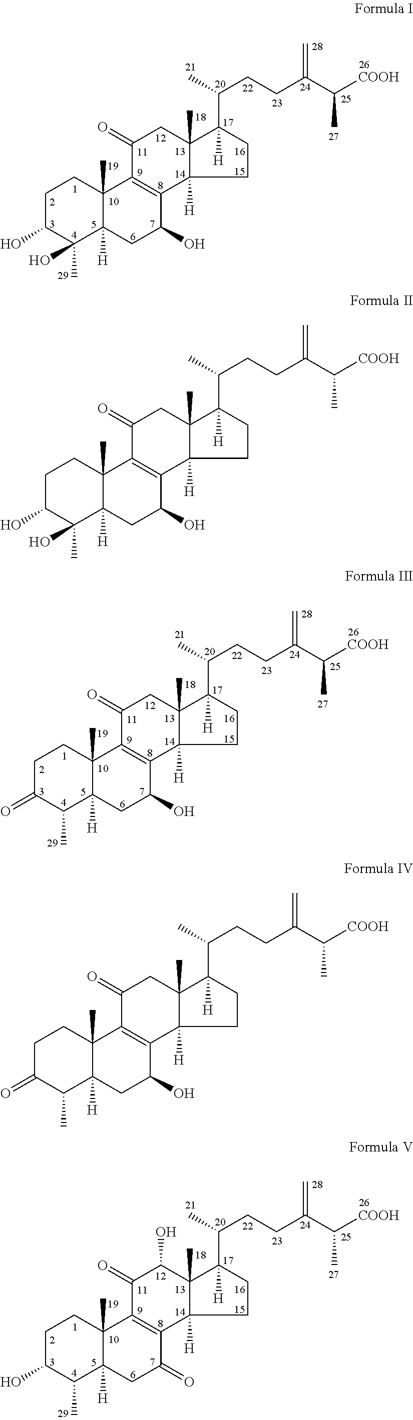

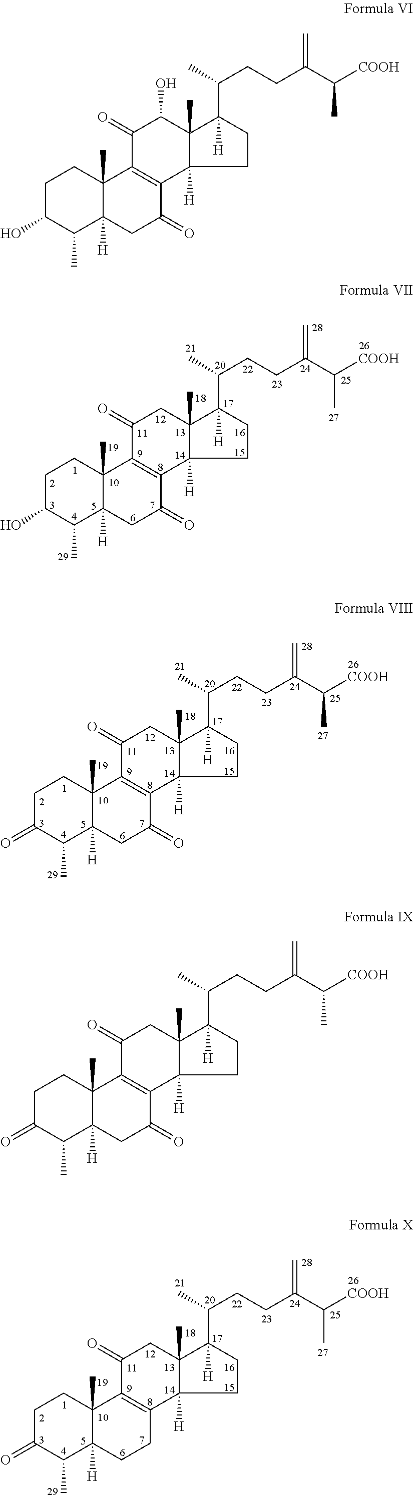

The TUPAC nominations and the structural formulas of the lanostane triterpenoid compounds L1 to L4 (formulas XI to XIV) extracted in the present invention are listed below in detail.

| Lanostane triterpenoid | Structural formula | Nomination |
|---|---|---|
| L1 | XI | dehydrosulphurenic acid |
| L2 | XII | sulphurenic acid |
| L3 | XIII | dehydroeburicoic acid |
| L4 | XIV | eburicoic acid |

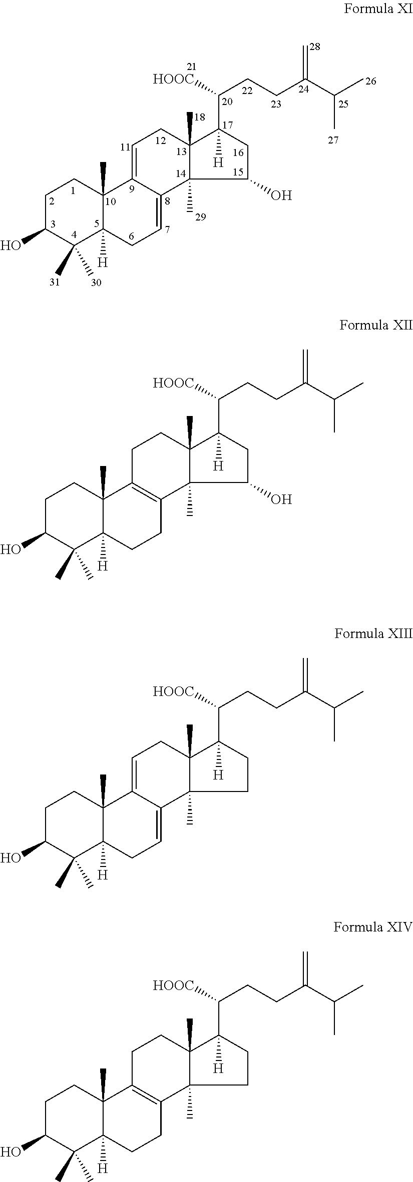

### Experiment 1

### The Analysis of the NMR Spectrum

Triterpenoid compounds are the main secondary metabolites of AC, which are divided into two types, i.e. ergostane and lanostane. The absolute amounts of the total ergostane triterpenoid compounds and the total lanostane triterpenoid compounds in the extract of AC are analyzed using NMR in the present invention (where the EtOH extract of the fruiting body of AC is an example but the example is not limited to it).

The experimental procedure for the detection is described as follows. First, an appropriate deuterium solvent is selected, and then the standards for these two types of compounds are respectively chosen to prepare the calibration standard curves with different concentrations. The certain amount of the internal standard is added to the standard, and the integral area ratio of the characteristic signal of each standard to the target signal of the internal standard is calculated. The integral value vs. the concentration is described using linear regression, and thus the calibration curve for the standards of the two types of compounds are obtained. Next, a specific concentration of the EtOH extract of the fruiting body of AC is prepared, and the equal amount of the deuterium solvent and the internal standard is added to perform the NMR spectrum analysis. After the characteristic signals of the two types of compounds and the target signal of the internal standard are integrated and the integral ratio is calculated, and the absolute total amount of the two types of compounds in the EtOH extract of the fruiting body of AC is obtained based on the calibration curve.

The analysis of the absolute amount of the total ergostane triterpenoid compounds and the total lanostane triterpenoid compounds in the EtOH extract of the fruiting body of AC is performed using NMR spectrum analysis in the present invention. The experimental conditions are listed as follows. The standards for the two types of compounds with different concentrations are prepared, which respectively are zhankuic acid A (a ergostane triterpenoid) and dehydroeburicoic acid (a lanostane triterpenoid). The internal standard (pyrazine, 0.132 mg) is added and dissolved in the DMSO-d6 solution (0.6 ml), which is the optimum deuterated solvent for the NMR spectrum analysis. The NMR apparatus is a Varian UNITY plus 400 MHz spectrometer, the scanning times are 10 (for 7 minutes), the spectrum width is 6002.4 Hz, and the width for impulse strength is 6.3 μs. The test solvent may be but is not limited in the DMSO-d6 solution, CDCl₃, C₅D₅N and so on. Please refer to Tables 1 and 2, the start point and the end point for the C-28 methylene characteristic signal of the standard for the two types of compounds are manually selected so as to calculate the integral area of the peak, and the integral area ratio of the C-28 methylene characteristic signal of the standard to the target signal of the internal standard pyrazine (δ_{H} 8.66) is calculated. The characteristic proton (the C-28 methylene) absorption signal of the standard (zhankuic acid A) is situated at δ_{H} 4.82 (2H, br d), and those of the standard (dehydroeburicoic acid) are situated at δ_{H} 4.63 (1H, s) and 4.70 (1 H, s). The experiment is made in triplicate and the relative standard deviation value (RSD %) is determined.

Please refer to Table 3, the integral ratio vs. the concentration are described by the linear regression, and the calibration curve (the standard curve and the coefficient of determination of the regression analysis) for the standards of the two types of compounds is obtained to use in this quantitative analysis method.

### Experiment 2

### The Design of the Extraction Method for the Ergostane and Lanostane Triterpenoid Compounds in the Fruiting Body of AC

The dried fruiting body of AC is ground into fine powder or is cut into fragments, which are added to 95% (v/v) EtOH solution at 75° C. at a ratio of 1:10 to 1:20 (weight/volume) to heat reflux and/or ultrasonic oscillate for 2 hours. The extract is precipitated at 4° C. overnight after cooling. The supernatant is filtered from the extract using a filter, and the precipitate is removed by centrifuging at 3,000 rpm for 30 minutes. The extract (i.e. the EtOH extract of the fruiting body of AC) is lyophilized and stored at −70° C.

It is known from the NMR spectrum analysis in Experiment 1 that the integral area ratio of the total ergostane triterpenoid compounds and the total lanostane triterpenoid compounds in the EtOH extract, and thus the concentrations of the total ergostane triterpenoid compounds and the total lanostane triterpenoid compounds are calculated.

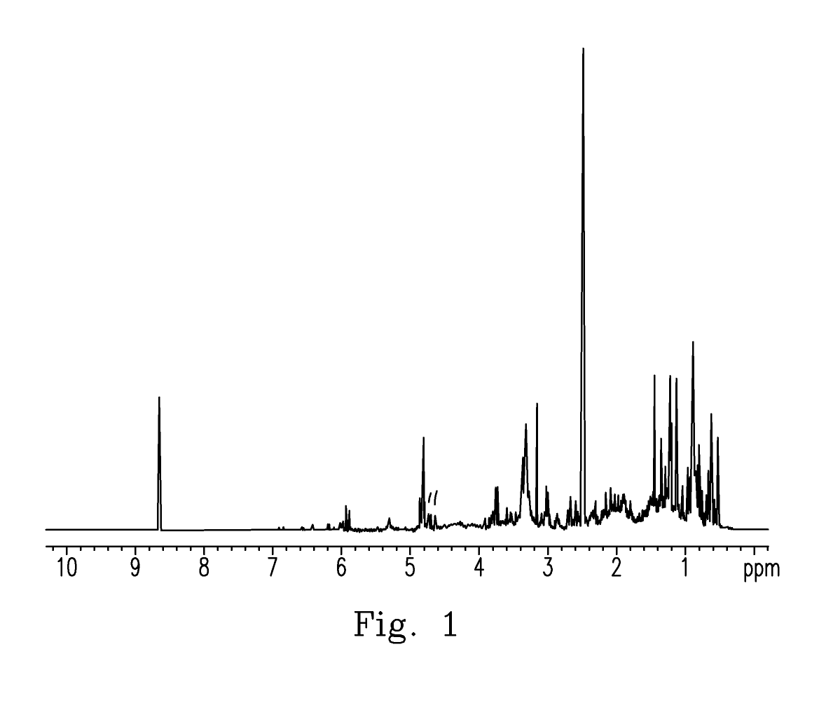

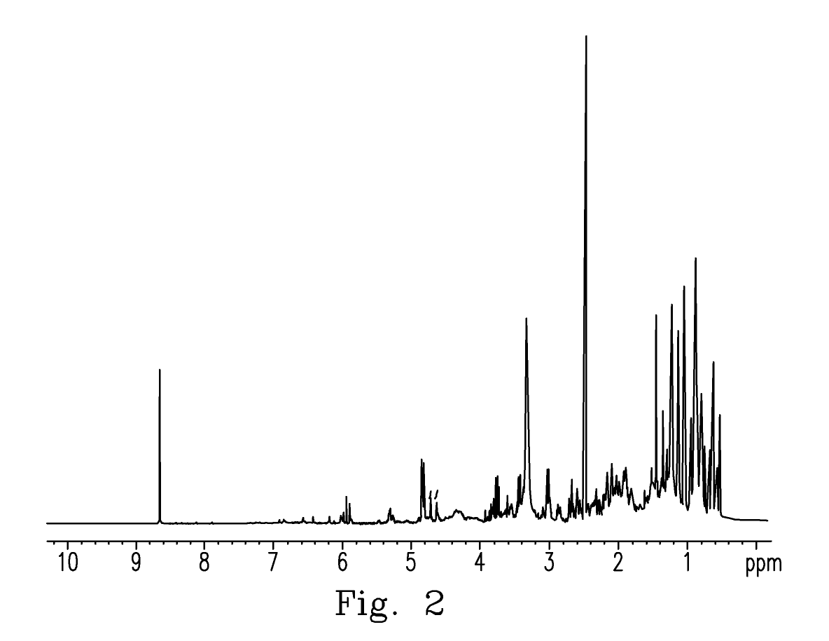

Please refer to FIGS. 1 and 2, which respectively are ¹H NMR spectra of the total triterpenoids in the EtOH extract of AC which are extracted using the heat reflux and the ultrasonic oscillation. The preliminary experiments are performed using these two different extraction methods in the present invention, and it is known from the NMR spectra that the integral area values of the characteristic signals for the total ergostane and the total lanostane triterpenoid compounds using these two extraction methods are not significantly different, proving that it has relevance to analyze the concentrations of the total ergostane and the total lanostane triterpenoid compounds in the fruiting body of AC using the NMR spectrum analysis.

### Experiment 3

### The Design of the Extraction Parameters for the Concentrations of the Ergostane and the Lanostane Triterpenoid Compounds in the Fruiting Body of AC

In the present invention, the extraction parameters that influences the concentration of the ergostane and the lanostane triterpenoid compounds in the fruiting body of AC are analyzed using mathematics and statistics to act as the reference/basis of control or modification. According to the actual data from the preliminary experiments, the mathematical model of the interrelationship between the parameters is established, and the extremes (including the maximum and the minimum) are found according to the model.

In the present invention, three domain values (−1, 0 and +1) for each extraction parameter are established using the model of the experimental design, and the optimal extraction conditions are surveyed, indicating that the range must include the maximum and the minimum. Three independent extraction parameters in the experiments of the present invention are a first parameter (the temperature), a second parameter (time) and a third parameter (the concentration of EtOH) to evaluate the optimal extraction procedure. The relative response value for the extraction condition is determined based on the integral area values of the characteristic signals for the total ergostane and the total lanostane triterpenoid compounds in the EtOH extract of the fruiting body of AC using the NMR spectrum analysis. It is known from the preliminary experiments that there are maximum and minimum values for each of three control parameters, and there are 16 different extraction conditions (the permutations of the domain values −1, 0 and +1) designed into the model for mathematics and statistics, wherein the experimental condition that the center point is passed through 6 times (0) means “the lack of fit” for the design of the model and the error information (referring to Tables 4 and 5).

It should be noted that three parameters and their maximum, minimum and an intermediate value between the maximum and the minimum are the control parameters for the optimal extraction procedure for AC. The skilled person in the art can sufficiently comprehend that it is practicable to use multiple parameters (such as 2, 3, 4 or more) as the control parameters. Furthermore, the multiple parameters are not limited to the temperature, time and the concentration of EtOH. The parameters involved in the extraction of AC can be control parameters, such as the pressure, the grinding level of AC when fragmented, the weight-volume ratio of AC in a solution, and so on. In addition to the maximum, the minimum and an intermediate value between the maximum and the minimum for each parameter acting as the control parameters for the optimal extraction procedure of AC, the maximum, the minimum and any value between the maximum and the minimum also can be the control parameters.

### Experiment 4

### The Optimal Extraction Parameters for the Concentrations of the Ergostane and the Lanostane Triterpenoid Compounds in the Fruiting Body of AC

The fine powder (70 mg) of the dried fruiting body of AC is weighed to perform the extraction using one of 16 different extraction conditions and ultrasonic oscillation. The extracts obtained under the 16 conditions are dried using a rotary evaporator. Each extract (6 mg) is added to the same amount of deuterated solvent and an internal standard (where 0.132 mg of the internal standard, pyrazine, is dissolved in 0.6 mL DMSO-d6) to perform the NMR spectrum analysis, and the integral area values of the characteristic signals for the total ergostane and the total lanostane triterpenoids in the EtOH extract of AC under different conditions are evaluated.

Please refer to Table 6, which describes the response values obtained from 16 different extraction conditions determined by NMR spectra. It can be seen from Table 6 that the optimal integral area values for the total ergostane and the total lanostane triterpenoid compounds respectively are 40.33 and 13.59 under the extraction conditions (i.e. the temperature: 50° C., time: 30 minutes, and the concentration of EtOH: 95%).

The variations in the extraction response values responding to the independent parameters are calculated using the above results and multiple regression analysis represented by the following formula I:

y=A₀+A₁x₁+A₂x₂+A₃x₃+A₁₂x₁x₂+A₁₃x₁x₂+A₂₃x₂x₃+A₁₁x₁²+A₂₂x₂²+A₃₃x₃² (I),

where y represents the extraction response value, A(0, 1, 2, 3, 12, 13, 23, 11, 22, 33) represent respective constants, and x(1, 2, 3) represent the independent control parameters. The linearity, quadratic term, cross product and P value (significance) are included in Table 7. The variability analysis reveals that the coefficients of determination (R²) for the total ergostane and the total lanostane triterpenoids are larger than 0.90, i.e. 0.97 and 0.94. The data prove that about 95% of the response values (variability) generated by the independent control parameter or between the parameters can be sufficiently explained by the established model. Furthermore, “the lack of fit” for the model is used to respond to the deficiency of the experimental design. The results reveal that the P values for the total ergostane and the total lanostane triterpenoids are larger than 0.05, i.e. 0.09 and 0.61, indicating that the designed model in the present invention can sufficiently and precisely predict the variability of the reaction. In addition, it can be seen from the regression analysis that the P values for the total ergostane and the total lanostane triterpenoids under the conditions designed for this model are smaller than 0.001 (referring to Table 8). Therefore, the designed model in the present invention are very accurate and reasonable for the present data. In the experimental design of the present invention, the response values obtained from 16 conditions can all be sufficiently explained using the multiple regression formula, and it is practicable to predict the variations of the amount of the total ergostane and total lanostane triterpenoids of AC under the different extraction parameters using the established regression mode.

The aforementioned formula (I) is a formula for multiple regression analysis with three extraction parameters. It can be comprehended by the skilled person in the art that the formula (I) and its analytic and statistic methodology can be adequately revised and modified when two, four or more extraction parameters are used in the multiple regression analysis, and the revision and modification fail in the scope of the claims of the present invention.

In addition to evaluating the reasonability and the lack of fit for the data obtained from the established model, independent control parameters and the interrelationship between the independent control parameters are analyzed (referring to Table 8). Regarding the independent parameters for the ergostane triterpenoids, the P value for the third parameter (the concentration of EtOH) is smaller than 0.01, and the P values for a cross product of monomial for other independent parameters (the temperature, time and the concentration of EtOH) and the square of the monominal for other independent parameters (the temperature, time and the concentration of EtOH) are larger than 0.05, indicating that the concentration of EtOH is a key control parameter for extracting the ergostane triterpenoids of AC. It is the same with the independent parameters for the lanostane triterpenoids, and the P value for the third parameter (the concentration of EtOH) is smaller than 0.01. In addition, the P value for the square (X₁²) of the first parameter (the temperature) is smaller than 0.05, indicating that the temperature is also a significant control parameter and the concentration of EtOH is the key control parameter for extracting the lanostane triterpenoids of AC.

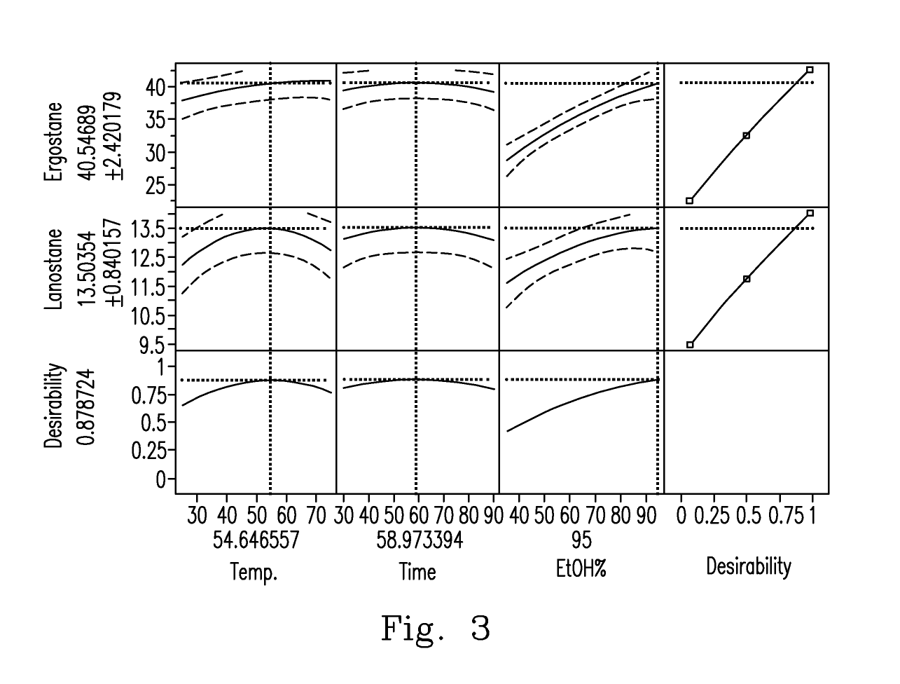

In the present invention, the extraction procedures for the triterpenoids of the fruiting body of AC is a verification mode, accompanied by the model design using mathematics and statistics, to obtain three predicted values (the temperature: 54.6° C., time: 58.9 minutes, and the concentration of EtOH: 95%) for the optimal extraction procedure of the triterpenoid compounds of AC (referring to FIG. 3). The key control parameter of the extraction procedure is the concentration of EtOH, and the P values for the ergostane and lanostane triterpenoids are smaller than 0.05. It can be seen from Table 8 that another significant control parameter for the lanostane triterpenoid is the square of the first parameter (the temperature), where its P value is 0.014. The largest predicted value for the optimal procedure under the condition (54.6° C., 58.9 minutes and 95% of the concentration of EtOH) can be obtained using the designed model of the present invention. That is, the highest extractability for the lanostane triterpenoids can be reached when the temperature is close to 55° C.; and the extractability will be reduced if the temperature continues to raise or fall. The parameter, “time”, is a factor that does not present statistical significance for the two types of compounds because the ultrasonic oscillation increases the extractability. It is found in the present invention that the extraction reaches a saturation state after 60 minutes of ultrasonic oscillation.

Therefore, the skilled person in the art can extract a small amount of AC according to Experiments 3 and 4 and the extraction parameters, find the optimal parameters for the extraction procedure or the parameters for the extraction procedure on the user's demand according to the NMR spectrum analysis and the multiple regression analysis, and then extract a large amount of AC using the optimal parameters for the extraction procedure or the parameters for the extraction procedure on the user's demand. Therefore, the obtained extract of AC contains the optimal ratio of the total ergostane to the total lanostane triterpenoid compounds, the most abundant amounts of the total ergostane and the total lanostane triterpenoid compounds, or the ratio or amount that the user requires.

### Experiment 5

### The Analysis of the Absolute Amount of the Total Ergostane and the Total Lanostane Triterpenoid Compounds

After the integral ratio for two types of compounds of the extract which is obtained from the optimal procedure is calculated using the calibration curve, the absolute amount of the total ergostane triterpenoid compounds is 513±0.18 μg/mg and that of the total lanostane triterpenoid compounds is 187±0.25 μg/mg of EtOH extract of the fruiting body of AC. A fast, energy-saving and efficient extraction procedure is established by the statistical model in the present invention, so that the extractability of the ergostane and lanostane triterpenoids of AC is increased.

### Experiment 6

### The HPLC Analysis of the Ergostane Triterpenoid Compounds and the Lanostane Triterpenoid Compounds

Because the chemical structures of the ergostane and the lanostane triterpenoids which are specifically included in the fruiting body of AC have a carboxyl group, a better chromatography result will be obtained in an acidic mobile phase. It had been determined that a better HPLC spectrum for the ergostane and lanostane triterpenoids of the EtOH extract of the fruiting body of AC can be obtained when the EtOH extract is in acetonitrile (CH₃CN)—H₂O (0.1% organic acid) or MeOH—H₂O (0.1% organic acid). The ergostane triterpenoid stereoisomeric mixture cannot be completely isolated although the retention time for the ergostane and lanostane triterpenoid compounds can be determined at the same time under this condition. To obtain a better isolation and resolution, the dissociation constant of the triterpenoid compound is further analyzed in the present invention, and then the acidity coefficient of each ergostane and lanostane triterpenoid compound (including 12 ergostane triterpenoid compounds E1-E12 and 4 lanostane triterpenoid compounds L1˜L4) is calculated using the online chemical algorithm software “SPARC (Sparc Performs Automated Reasoning in Chemistry)” (referring to Table 9). The acidity coefficients of these two types of triterpenoid compounds range between 4.40 4.60. Next, five pH values (i.e. 3.75, 4.00, 4.25, 4.50 and 5.00) for the mobile phase are prepared to perform the HPLC and then the HPLC spectra are analyzed. To look for the optimal chromatographic conditions, the analytic result is evaluated according to the resolution (Rs) of each triterpenoid compound in its chromatography spectrum.

The conditions of HPLC are described as follows. The HPLC apparatus is the Agilent 1200 HPLC system (Agilent Technologies), the detector is an API 4000 Triple Quadrupole spectrometer (Applied Biosystem, Foster City, Calif., USA), the HPLC column is Agilent EC-C₁₈ (150×4.6 mm), and solvents A and B in the mobile phase are CH₃CN and pure water (HPLC grade H₂O supplemented with 0.1% acetic acid and ammonium acetate (10 mM) to final pH of 3.75, 4.00, 4.25, 4.50 and 5.00). Flow rate is 1.3 ml/min. The temperature of the column is room temperature, and the detection wavelength is UV 254 nm. The conditions of the solvent system are described as follows. Mobile phase includes solvents A and B, and linear gradients are 0˜15 min (39% A˜44% A), 15˜17.5 min (44% A˜45% A), 17.5˜22.5 min (45% A˜47% A), 22.5˜27.5 min (47% A˜50% A), 27.5˜30 min (50% A˜53% A) and 30˜35 min (53% A˜55% A), 35˜45 min (55% A 65% A), 45˜55 min (65% A˜98% A) and 55˜60 min (98% A˜100% A). The Flow rate and the temperature of the column are the same as above.

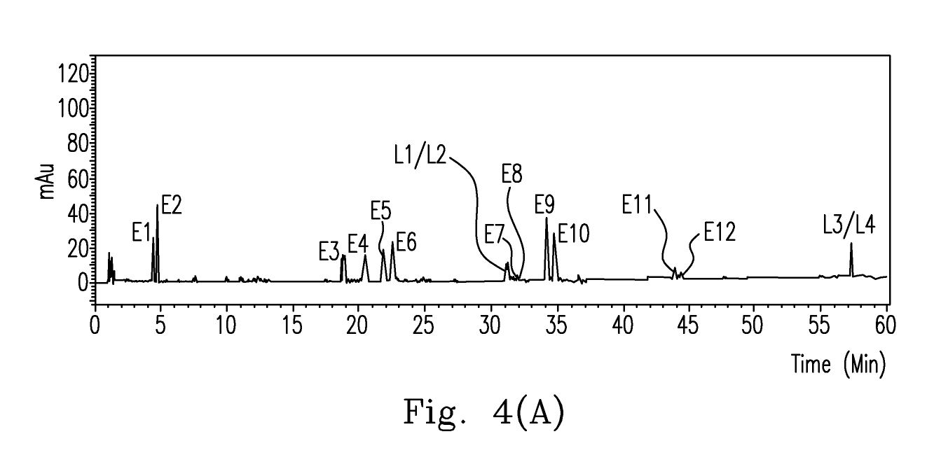

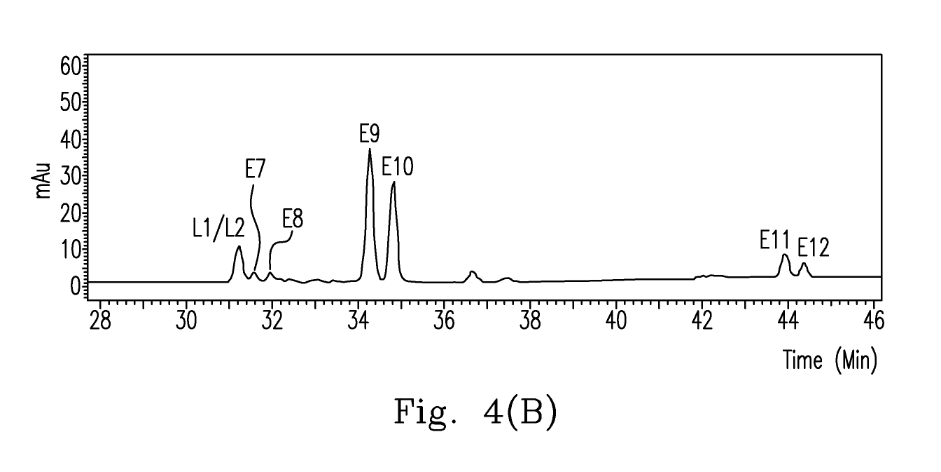

The results show that better resolution for each ergostane and lanostane triterpenoid compound will be obtained when the pH of the mobile phase is 4.25 (referring to FIGS. 4(A) and 4(B)). In the optimal HPLC spectrum, two ergostane triterpenoid compounds in a pair have better resolution and isolation effects, and thus can be applied in the quantitative analysis of HPLC-tandem MS (such as the triple quadrupole spectrometer). Furthermore, the sample includes 12 ergostane and 4 lanostane triterpenoid compounds. Although the spectrum only shows the signals for two lanostane triterpenoid compounds (i.e. dehydrosulphurenic acid (L1) and dehydroeburicoic acid (L3)), compounds L1 and L2 (sulphurenic acid) have similar structures, compound L3 and L4 (eburicoic acid) have similar structures, and the structural difference between the group 1 (L1 and L3) and the group 2 (L2 and L4) is that group 1 has two set of double bonds (C7-C8 and C9-C11) and group 2 has one set of double bonds (C8-C9). Although the peaks of the compounds L1 and L2 overlap and the peaks of the compounds L3 and L4 overlap, their molecular weights (Mw) are different. The qualitative and quantitative determination of the lanostane triterpenoid compounds can be performed under the above optimized HPLC conditions using the HPLC-tandem MS and the property that the Mws are different.

### Experiment 7

### The Analysis for the Amount of Each Ergostane Triterpenoid Compound and Each Lanostane Triterpenoid Compound

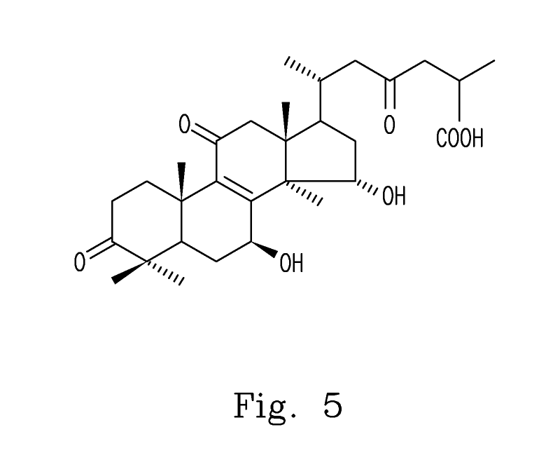

Furthermore, the quantitative analysis for the 16 triterpenoid compounds (E1 E12 and L1-L4) in the EtOH extract of the fruiting body of AC is performed using the HPLC-tandem MS, the detector is the triple quadrupole spectrometer with high quantification precision, and the ionic scanning mode is used for multiple reaction monitoring (MRM). In this experiment, ganoderic acid A with the Mw of 516 (FIG. 5) is the internal standard, which has similar physical, chemical and chromatographic properties with 16 triterpenoid compounds. The liquid chromatography/mass spectrometer (LC-MS/MS) is an Agilent 1200 HPLC system and the API 4000 Triple Quadrupole spectrometer, and the ionization source for detection is the electrospray ionization (ESI) accompanied by the negative ion mode.

Two pairs of daughter ions are selected from 16 triterpenoid compounds (E1˜E12 and L1˜L4) and the internal standard (ganoderic acid A), the daughter ion at the first strength acts as the quantitative ion, and the daughter ion at the second strength acts as the qualitative ion. Please refer to Table 10, which are the results for the optimal mass spectrum parameters, the Extracted Ion Chromatogram (XIC) and the spectra for the daughter ions of the 16 triterpenoid compounds. To detect the 16 triterpenoid compounds simultaneously, each standard with 5 different concentrations (10˜1000 ng/ml) is prepared, the integral ratio of each compound at each concentration to the quantitative ion of the internal standard is calculated, and the calibration curve is made. The results in Table 10 show that the determination coefficient (R′) of the linear regression for each of the 16 triterpenoid compounds is above 0.99. Table 11 shows the individual amounts of the 16 triterpenoid compounds in the EtOH extract of AC determined using the quantitative method of HPLC-MS.

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

TABLE 1
The integral area ratio of the C-28 methylene signal for
zhankuic acid A in the different levels of the ergostane
triterpenoid standard to the target signal of an internal
standard, and its relative standard deviation

| | Integral area ratio | | | | |
|---|---|---|---|---|---|
| Weight (mg) | 1 | 2 | 3 | Average | RSD % |
| 2.020 | 28.07 | 28.47 | 28.58 | 28.37 | 0.95 |
| 3.030 | 38.61 | 38.68 | 38.75 | 38.68 | 0.18 |
| 4.000 | 49.31 | 48.74 | 49.95 | 49.33 | 1.23 |
| 5.040 | 63.82 | 63.99 | 64 | 63.94 | 0.16 |
| 6.060 | 75.8 | 75.39 | 75.11 | 75.43 | 0.46 |

TABLE 2
The integral area ratio of the C-28 methylene signal for
dehydroeburicoic acid in the different levels of the lanostane
triterpenoid standard to the target signal of the internal
standard, and its relative standard deviation

| | Integral area ratio | | | | |
|---|---|---|---|---|---|
| Weight (mg) | 1 | 2 | 3 | Average | RSD % |
| 1.150 | 13.54 | 13.4 | 13.49 | 13.48 | 0.53 |
| 2.100 | 24.44 | 24.07 | 24.68 | 24.40 | 1.26 |
| 3.050 | 35.39 | 34.91 | 34.56 | 34.95 | 1.19 |
| 4.040 | 46.58 | 46.01 | 46.37 | 46.32 | 0.62 |
| 5.010 | 55.77 | 55.87 | 55.6 | 55.75 | 0.24 |

TABLE 3
Calibration curves of zhankoic acid A and dehydroeburicoic acid

| Compound | Calibration curve | Coefficient of determination |
|---|---|---|
| Zhankuic acid A | Y = 11.8 X + 3.5 | 0.997 |
| Dehydroeburicoic acid | Y = 11.0 X + 1.1 | 0.999 |

TABLE 4
Three independent extraction parameters and their domain values for
the extraction procedure of the triterpenoids of A. cinnamomea

| Domain value | Temperature (° C.) | Time (minute) | EtOH (%) (v/v) |
|---|---|---|---|
| −1 | 25 | 30 | 35 |
| 0 | 50 | 60 | 65 |
| 1 | 75 | 90 | 95 |

TABLE 5
Sixteen (16) conditions for the extraction procedures of the triterpenoids
of A. cinnamomea designed using mathematics and statistics

| | Actual parameters | | |
|---|---|---|---|
| Examples | Temperature (° C.) | Time (minute) | EtOH (%) (v/v) |
| 1 | 25 | 30 | 35 |
| 2 | 25 | 30 | 95 |
| 3 | 25 | 60 | 65 |
| 4 | 25 | 90 | 35 |
| 5 | 25 | 90 | 95 |
| 6 | 50 | 30 | 65 |
| 7 | 50 | 60 | 35 |
| 8 | 50 | 60 | 65 |
| 9 | 50 | 60 | 65 |
| 10 | 50 | 60 | 95 |
| 11 | 50 | 90 | 65 |
| 12 | 75 | 30 | 35 |
| 13 | 75 | 30 | 95 |
| 14 | 75 | 60 | 65 |
| 15 | 75 | 90 | 35 |
| 16 | 75 | 90 | 95 |

TABLE 6
The integral area values of the total ergostane and the total lanostane
triterpenoids for 16 extraction conditions determined by NMR

| | Actual parameter value | | | Response value (Y) | |
|---|---|---|---|---|---|
| | Temp | Time | EtOH % | Total ergostane | total lanostane |
| No. | (° C.) | (min) | (v/v) | triterpenoids | triterpenoids |
| 1 | 25 | 30 | 35 | 27.58 | 9.63 |
| 2 | 25 | 30 | 95 | 37.18 | 11.81 |
| 3 | 25 | 60 | 65 | 34.52 | 11.43 |
| 4 | 25 | 90 | 35 | 26.94 | 9.74 |
| 5 | 25 | 90 | 95 | 36.81 | 12.07 |
| 6 | 50 | 30 | 65 | 35.56 | 12.64 |
| 7 | 50 | 60 | 35 | 30.2 | 11.89 |
| 8 | 50 | 60 | 65 | 34.31 | 12.81 |
| 9 | 50 | 60 | 65 | 34.57 | 12.17 |
| 10 | 50 | 60 | 95 | 40.33 | 13.59 |
| 11 | 50 | 90 | 65 | 35.15 | 12.87 |
| 12 | 75 | 30 | 35 | 26.38 | 10.67 |
| 13 | 75 | 30 | 95 | 39.43 | 12.42 |
| 14 | 75 | 60 | 65 | 36.91 | 12.88 |
| 15 | 75 | 90 | 35 | 24.22 | 10.22 |
| 16 | 75 | 90 | 95 | 39.28 | 12.01 |

TABLE 7
Deviation analysis for the temperature, time and concentration of EtOH in the EtOH
extract of the fruiting body of A. cinnamomea

| | Total ergostane triterpenoids | | | | Total lanostane triterpenoidns | | | |
|---|---|---|---|---|---|---|---|---|
| | | Sum of | | | | Sum of | | |
| Item | DF | squares | F ratio | P value | DF | squares | F ratio | P value |
| Temp.(25,75) | 1 | 1.01761 | 0.5458 | 0.4879 | 1 | 1.23904 | 5.515 | 0.0572 |
| Time (30,90) | 1 | 1.39129 | 0.7463 | 0.4208 | 1 | 0.00676 | 0.0301 | 0.868 |
| EtOH % (35,95) | 1 | 333.04441 | 178.6439 | <.0001 | 1 | 9.50625 | 42.3126 | 0.0006 |
| Temp.*Temp. | 1 | 0.21125 | 0.1133 | 0.7479 | 1 | 0.1891125 | 0.8417 | 0.3943 |
| Temp.*EtOH % | 1 | 9.3312 | 5.0052 | 0.0666 | 1 | 0.1176125 | 0.5235 | 0.4966 |
| Time*EtOH % | 1 | 0.6498 | 0.3486 | 0.5765 | 1 | 0.0045125 | 0.0201 | 0.8919 |
| Temp.*Temp. | 1 | 2.07937 | 1.1154 | 0.3316 | 1 | 2.6182132 | 11.6537 | 0.0143 |
| Time*Time | 1 | 4.10683 | 2.2029 | 0.1883 | 1 | 0.4145768 | 1.8453 | 0.2232 |
| EtOH %*EtOH % | 1 | 4.72046 | 2.532 | 0.1627 | 1 | 0.4465336 | 1.9875 | 0.2083 |
| ANOVA | | | | | | | | |
| Model | 9 | 375.04354 | 22.3524 | 0.0006 | 9 | 19.40214 | 9.5955 | 0.0062 |
| Error | 6 | 11.18576 | | | 6 | 1.348004 | | |
| C. Total | 15 | 386.22929 | | | 15 | 20.750144 | | |
| Lack of fit | | | | | | | | |
| Lack of fit | 5 | 11.151956 | 65.9879 | 0.0932 | 5 | 1.1432039 | 1.1164 | 0.6126 |
| Pure error | 1 | 0.0338 | | | 1 | 0.2048 | | |
| Total error | 6 | 11.185756 | | | 6 | 1.3480039 | | |
| Summary of fit | | | | | | | | |

| | | |
|---|---|---|
| R² | 0.971039 | 0.935036 |
| R²-adjustmenmt | 0.927596 | 0.837591 |

DF: Degree of freedom

TABLE 8
Regression analysis and significance for the individual control
parameters in the ergostane and lanostane triterpenoids

| | Total ergostane | | Total lanostane | |
|---|---|---|---|---|
| | triterpenoids | | triterpenoids | |
| | Regression | | Regression | |
| Term^{a} | coefficients | P-values | coefficients | P-values |
| Intercept | 35.882069 | <.0001^{b} | 12.931034 | <.0001^{b} |
| Temp(25, 75) | 0.319 | 0.4879 | 0.352 | 0.0572 |
| Time(30, 90) | −0.373 | 0.4208 | −0.026 | 0.868 |
| EtOH %(35, 95) | 5.771 | <.0001^{b} | 0.975 | 0.0006^{b} |
| Temp*Time | −0.1625 | 0.7479 | −0.15375 | 0.3943 |
| Temp*EtOH % | 1.08 | 0.0666 | −0.12125 | 0.4966 |
| Time*EtOH % | 0.285 | 0.5765 | 0.02375 | 0.8919 |
| Temp*Temp | −0.888103 | 0.3316 | −0.996552 | 0.0143^{b} |
| Time*Time | −1.248103 | 0.1883 | −0.396552 | 0.2232 |
| EtOH %*EtOH % | −1.338103 | 0.1627 | −0.411552 | 0.2083 |

^{a}Temperature (X₁), Time (X₂) and EtOH % (X₃).
^{b}P < 0.05 indicates statistical significance.

TABLE 9
Acidity coefficient (pKa) of the ergostane and lanostane compounds calculated by the
online chemical algorithm software “SPARC Performs Automated Reasoning in Chemistry”

| | | | Acidity |
|---|---|---|---|
| | | | coefficient |
| Index | Compound | Structure | (pKa) |
| 1 | E1 | C[C@]34CC(═O)C1═C([C@]([H])(C[C@@]2([H])[C@@](C)(O[H])[C@@]([H])(CC[C@] | 4.45 |
| | | 12C)O[H])O[H])[C@]3([H])CC[C@]4([H])[C@@](C)([H])CCC(═C)[C@@](C)([H])C(═O) | |
| | | O[H] | |
| 2 | E2 | C[C@]34CC(═O)C1═C([C@]([H])(C[C@@]2([H])[C@@](C)(O[H])[C@@]([H])(CC[C@] | 4.45 |
| | | 12C)O[H])O[H])[C@]3([H])CC[C@]4([H])[C@@](C)([H])CCC(═C)[C@](C)([H])C(═O)O[ | |
| | | H] | |
| 3 | E3 | O═C2CC[C@]1(C)C4═C([C@]([H])(C[C@@]1([H])[C@@]2(C)[H])O[H])[C@]3([H])CC[ | 4.45 |
| | | C@@]([H])([C@@]3(C)CC4═O)[C@@](C)([H])CCC(═C)[C@@](C)([H])C(═O)O[H] | |
| 4 | E4 | O═C2CC[C@]1(C)C4═C([C@]([H])(C[C@@]1([H])[C@@]2(C)[H])O[H])[C@]3([H])CC[ | 4.45 |
| | | C@@]([H])([C@@]3(C)CC4═O)[C@@](C)([H])CCC(═C)[C@](C)([H])C(═O)O[H] | |
| 5 | E5 | O═C2C[C@@]1([H])[C@@](C)([H])[C@@]([H])(CC[C@]1(C)C4═C2[C@]3([H])CC[C@ | 4.45 |
| | | @]([H])([C@@]3(C)CC4═O)[C@@](C)([H])CCC(═C)[C@@](C)([H])C(═O)O[H])O[H] | |
| 6 | E6 | O═C2C[C@@]1([H])[C@@](C)([H])[C@@]([H])(CC[C@]1(C)C4═C2[C@]3([H])CC[C@ | 4.45 |
| | | @]([H])([C@@]3(C)CC4═O)[C@@](C)([H])CCC(═C)[C@](C)([H])C(═O)O[H])O[H] | |
| 7 | E7 | O═C2C[C@@]1([H])[C@@](C)([H])[C@@]([H])(CC[C@]1(C)C4═C2[C@]3([H])CC[C@ | 4.45 |
| | | @]([H])([C@@]3(C)CC4═O)[C@@](C)([H])CCC(═C)[C@@](C)([H])C(═O)O[H])O[H] | |
| 8 | E8 | O═C2C[C@@]1([H])[C@@](C)([H])[C@@]([H])(CC[C@]1(C)C4═C2[C@]3([H])CC[C@ | 4.45 |
| | | @]([H])([C@@]3(C)CC4═O)[C@@](C)([H])CCC(═C)[C@](C)([H])C(═O)O[H])O[H] | |
| 9 | E9 | O═C2CC[C@]1(C)C4═C(C(═O)C[C@@]1([H])[C@@]2(C)[H])[C@]3([H])CC[C@@]([H]) | 4.45 |
| | | ([C@@]3(C)CC4═O)[C@@](C)([H])CCC(═C)[C@@](C)([H])C(═O)O[H] | |
| 10 | E10 | O═C2CC[C@]1(C)C4═C(C(═O)C[C@@]1([H])[C@@]2(C)[H])[C@]3([H])CC[C@@]([H]) | 4.45 |
| | | ([C@@]3(C)CC4═O)[C@@](C)([H])CCC(═C)[C@](C)([H])C(═O)O[H] | |
| 11 | E11 | O═C2CC[C@]1(C)C4═C(CC[C@@]1([H])[C@@]2(C)[H])[C@]3([H])CC[C@@]([H])([C@ | 4.45 |
| | | @]3(C)CC4═O)[C@@](C)([H])CCC(═C)[C@@](C)([H])C(═O)O[H] | |
| 12 | E12 | O═C2CC[C@]1(C)C4═C(CC[C@@]1([H])[C@@]2(C)[H])[C@]3([H])CC[C@@]([H])([C@ | 4.45 |
| | | @]3(C)CC4═O)[C@@](C)([H])CCC(═C)[C@](C)([H])C(═O)O[H] | |
| 13 | L1 | C[C@]34CC═C1C(═CC[C@@]2([H])C(C)(C)[C@]([H])(CC[C@]12C)O[H])[C@]3(C)[C@]([ | 4.44 |
| | | H])(C[C@]4([H])[C@@]([H])(CCC(═C)C(C)(C)[H])C(═O)O[H])O[H] | |
| 14 | L2 | C[C@]34CCC1═C(CC[C@@]2([H])C(C)(C)[C@]([H])(CC[C@]12C)O[H])[C@]3(C)[C@]([ | 4.49 |
| | | H])(C[C@]4([H])[C@@]([H])(CCC(═C)C(C)(C)[H])C(═O)O[H])O[H] | |
| 15 | L3 | C[C@]34CC═C1C(═CC[C@@]2([H])C(C)(C)[C@]([H])(CC[C@]12C)O[H])[C@]3(C)[C@]([ | 4.3 |
| | | H])(C[C@]4([H])[C@@]([H])(CCC(═C)C(C)(C)[H])C(═O)O[H])OC(═O)C | |
| 16 | L4 | C[C@]34CCC1═C(CC[C@@]2([H])C(C)(C)[C@]([H])(CC[C@]12C)O[H])[C@]3(C)[C@]([ | 4.36 |
| | | H])(C[C@]4([H])[C@@]([H])(CCC(═C)C(C)(C)[H])C(═O)O[H])OC(═O)C | |
| 17 | L5 | C[C@]34CC═C1C(═CC[C@@]2([H])C(C)(C)[C@]([H])(CC[C@]12C)O[H])[C@]3(C)CC[C | 4.54 |
| | | @]4([H])[C@@]([H])(CCC(═C)C(C)(C)[H])C(═O)O[H] | |
| 18 | L6 | CC(C)([H])C(═C)CC[C@@]([H])(C(═O)O[H])[C@@]4([H])CC[C@@]3(C)C=2CC[C@@]1( | 4.59 |
| | | [H])C(C)(C)[C@]([H])(CC[C@]1(C)C═2CC[C@]34C)O[H] | |

TABLE 10
The optimized conditions for the mass spectrum and its determination coefficient (R²)
in regression of the 16 triterpenoid compounds in the EtOH extract of A. cinnamomea

| | Retention | Parent ion | | | |
|---|---|---|---|---|---|
| Analytes | time (t_{R}) | (m/z) [M − H] | Daughter ion (m/z) | Calibration curve equation | R² |
| E1 | 3.96 | 487 | 443^{a}/407 | Y = (0.00408 ± 0.00006) x − (0.00477 ± 0.00437) | 0.997 |
| E2 | 4.23 | 487 | 443^{a}/407 | Y = (0.00396 ± 0.00016) x + (0.00748 ± 0.00523) | 0.999 |
| E3 | 17.10 | 469 | 425^{a}/247 | Y = (0.01550 ± 0.00091) x − (0.18615 ± 0.04059) | 0.998 |
| E4 | 18.70 | 469 | 425^{a}/247 | Y = (0.00976 ± 0.00039) x − (0.12398 ± 0.02426) | 0.997 |
| E5 | 19.88 | 485 | 441^{a}/413 | Y = (0.00697 ± 0.00009) x + (0.05383 ± 0.02665) | 0.997 |
| E6 | 20.53 | 485 | 441^{a}/413 | Y = (0.00629 ± 0.00010) x − (0.02017 ± 0.00685) | 0.998 |
| E7 | 29.43 | 469 | 425^{a}/409 | Y = (0.00122 ± 0.00001) x − (0.02136 ± 0.00626) | 0.998 |
| E8 | 29.87 | 469 | 425^{a}/409 | Y = (0.00147 ± 0.00011) x − (0.02376 ± 0.01168) | 0.997 |
| E9 | 32.23 | 467 | 423^{a}/407 | Y = (0.02541 ± 0.00031) x + (0.19959 ± 0.15536) | 0.998 |
| E10 | 32.85 | 467 | 423^{a}/407 | Y = (0.03640 ± 0.00173) x − (0.22022 ± 0.11636) | 0.999 |
| E11 | 42.29 | 453 | 409^{a}/393 | Y = (0.00069 ± 0.00002) x − (0.00934 ± 0.00062) | 0.999 |
| E12 | 42.64 | 453 | 409^{a}/393 | Y = (0.00051 ± 0.00001) x − (0.01150 ± 0.00134) | 0.999 |
| L1 | 28.83 | 483 | 268/83^{a} | Y = (0.00070 ± 0.00002) x − (0.00718 ± 0.00375) | 0.997 |
| L2 | 29.86 | 485 | 355^{a}/83 | Y = (0.00023 ± 0.000002) x + (0.00614 ± 0.00116) | 0.997 |
| L3 | 55.70 | 467 | 371/337^{a} | Y = (0.00744 ± 0.00042) x + (0.14245 ± 0.12241) | 0.997 |
| L4 | 56.46 | 469 | 373/339^{a} | Y = (0.00169 ± 0.00012) x + (0.00628 ± 0.00410) | 0.998 |
| IS | 4.29 | 515 | 497^{a}/285 | | |

^{a}Quantification transitions

TABLE 11
The individual amounts (μg/mg) of the 16
triterpenoid compounds in the EtOH extract of
A. cinnamomea determined by HPLC-tandem MS

| | Range of | Concentration |
|---|---|---|
| | concentration | of sample |
| | (ppb) | (ppb) |
| 25(S)-antcin K (E1) | 10-1000 | 16.739 ± 0.38 |
| 25(R)-antcin K (E2) | 10-1000 | 34.539 ± 0.31 |
| 25(S)-antcin C (E3) | 10-1000 | 5.997 ± 0.30 |
| 25(R)-antcin C (E4) | 10-1000 | 10.751 ± 0.32 |
| 25(R)-zhankuic acid C (E5) | 10-1000 | 40.206 ± 0.35 |
| 25(S)-zhankuic acid C (E6) | 10-1000 | 54.288 ± 0.30 |
| zhankuic acid B (E7) | 10-1000 | 48.102 ± 0.45 |
| zhankuic acid B (E8) | 10-1000 | 39.012 ± 0.40 |
| 25(S)-zhankuic acid A (E9) | 10-1000 | 31.509 ± 0.25 |
| 25(R)-zhankuic acid A (E10) | 10-1000 | 22.251 ± 0.21 |
| antcin A (E11) | 10-1000 | 120.418 ± 0.53 |
| antcin A (E12) | 10-1000 | 144.023 ± 0.57 |
| dehydrosulphurenic acid (L1) | 10-1000 | 45.685 ± 0.40 |
| sulphurenic acid (L2) | 10-1000 | 38.561 ± 0.35 |
| dehydroeburicoic acid (L3) | 10-1000 | 26.724 ± 0.38 |
| eburicoic acid (L4) | 10-1000 | 2.897 ± 0.41 |

What is claimed is:

1. An extraction method for an Antrodia cinnamomea, comprising steps of:
(a) selecting a first parameter, a second parameter and a third parameter, wherein each of the first, the second and the third parameters has a maximum, a minimum and a value between the maximum and the minimum, and defining the minimum, the value and the maximum respectively as domain values −1, 0 and 1;
(b) forming a plurality of extraction conditions by permuting the domain values −1, 0 and 1 of all of the first, the second and the third parameters;
(c) after extracting a first amount of the A. cinnamomea using each of the plurality of extraction conditions, obtaining a plurality of first extracts of the A. cinnamomea;
(d) evaluating an extraction response value for each of the plurality of first extracts of the A. cinnamomea with formula I:
y=A₀+A₁x₁+A₂x₂+A₃x₃+A₁₂x₁x₂+A₁₃x₁x₂+A₂₃x₂x₃+A₁₁x₁²+A₂₂x₂²+A₃₃x₃² (I),
where y represents the extraction response value, A(0, 1, 2 . . . ) represent respective constants, and x(1, 2, 3) represent the respective first, second and third parameters;
(e) analyzing the plurality of first extracts of the A. cinnamomea with nuclear magnetic resonance (NMR) to obtain a first integral area value for a first characteristic signal of total ergostane triterpenoids and a second integral area value for a second characteristic signal of total lanostane triterpenoids in each of the plurality of first extracts of the A. cinnamomea; and
(f) extracting a second amount of the A. cinnamomea according to a user's demand based on the extraction response value, the first and the second integral area values and a specific one of the plurality of extraction conditions to obtain a second extract of the A. cinnamomea.

2. The extraction method according to claim 1, wherein the A. cinnamomea is one of a fruiting body of the A. cinnamomea, an mycelium of the A. cinnamomea and the combination thereof.

3. The extraction method according to claim 1, wherein the first, the second and the third parameters respectively are temperature, time and concentration of an alcohol solution.

4. The extraction method according to claim 3, wherein a maximum, a minimum and a value between the maximum and the minimum of the temperature respectively are 75° C., 25° C. and 50° C.

5. The extraction method according to claim 3, wherein a maximum, a minimum and a value between the maximum and the minimum of the time respectively are 90 minutes, 30 minutes and 60 minutes.

6. The extraction method according to claim 3, wherein the concentration of the alcohol solution is obtained by formulating an alcohol with water, and the alcohol is one of methanol and ethanol.

7. The extraction method according to claim 6, wherein the ethanol solution has a concentration obtained by formulating the ethanol with water when the alcohol is the ethanol, and a maximum, a minimum and a value between the maximum and the minimum of the concentration of the ethanol solution respectively are 95% (v/v), 35% (v/v) and 65% (v/v).

8. The extraction method according to claim 3, wherein extraction levels of the total ergostane triterpenoids and the total lanostane triterpenoids of the A. cinnamomea are the highest under the conditions of 54.6° C. for the temperature, 58.9 minutes for the time and 95% (v/v) of the concentration for the ethanol solution.

9. The extraction method according to claim 1, wherein the second amount is more abundant than the first amount.

10. An analytic method for optimizing parameters for an extraction procedure of an Antrodia cinnamomea, the analytic method comprising steps of:
(a) selecting a first parameter, a second parameter and a third parameter, wherein each of the first, the second and the third parameters has a maximum, a minimum and a value between the maximum and the minimum, and defining the minimum, the value and the maximum respectively as domain values −1, 0 and 1;
(b) forming a plurality of extraction conditions by permuting the domain values −1, 0 and 1 of all of the first, the second and the third parameters;
(c) after extracting an amount of the A. cinnamomea using each of the plurality of extraction conditions, obtaining a plurality of extracts of the A. cinnamomea;
(d) evaluating an extraction response value for each of the plurality of extracts of the A. cinnamomea with formula I:
y=A₀+A₁x₁+A₂x₂+A₃x₃+A₁₂x₁x₂+A₁₃x₁x₂+A₂₃x₂x₃+A₁₁x₁²+A₂₂x₂²+A₃₃x₃² (I),
where y represents the extraction response value, A(0, 1, 2 . . . ) represent respective constants, and x(1, 2, 3) represent the respective first, second and third parameters;
(e) analyzing the plurality of extracts of the A. cinnamomea with nuclear magnetic resonance (NMR) to obtain a first integral area value for a first characteristic signal of total ergostane triterpenoids and a second integral area value for a second characteristic signal of total lanostane triterpenoids in each of the plurality of extracts of the A. cinnamomea; and
(f) determining optimized parameters for the extraction procedure of the A. cinnamomea using the first and the second integral area values and the extraction response value.

11. The analytic method according to claim 10, wherein each of the total ergostane triterpenoids has a first parent ion, a first daughter ion with a first intensity and a second daughter ion with a second intensity, each of the total lanostane triterpenoids has a second parent ion, a third daughter ion with a third intensity and a fourth daughter ion with a fourth intensity, and the analytic method further comprises:
(g) analyzing the first and the second parent ions and the first, the second, the third and the fourth daughter ions of each of the plurality of extracts of the A. cinnamomea using high performance liquid chromatography/tandem mass spectrometer (HPLC-tandem MS).

12. The analytic method according to claim 11, further comprising:
(h) calculating a level of each of the total ergostane triterpenoids and a level of each of the total lanostane triterpenoids.

13. An analytic method for optimizing parameters of an extraction procedure of an Antrodia cinnamomea, the analytic method comprising steps of:
(a) selecting a plurality of parameters, wherein each of the parameters has a maximum, a minimum and a value between the maximum and the minimum, and the minimum, the value and the maximum respectively are defined as domain values −1, 0 and 1;
(b) forming a plurality of extraction conditions by permuting the domain values −1, 0 and 1 of all of the plurality of parameters;
(c) after extracting an amount of the A. cinnamomea using each of the plurality of extraction conditions, obtaining a plurality of extracts of the A. cinnamomea; and
(d) calculating an extraction response value for each of the plurality of extracts of the A. cinnamomea using multiple regression to determine an optimized parameter for the extraction procedure.

14. The analytic method according to claim 13, wherein step (d) further comprises:
(d1) performing nuclear magnetic resonance (NMR) on each of the plurality of extracts to obtain an integral area value for a characteristic signal of total triterpenoids in each of the plurality of extracts of the A. cinnamomea.

15. The analytic method according to claim 14, wherein step (d1) further comprises:
(d2) determining the optimized parameter for the extraction procedure according to the integral area value and the extraction response value.
